(19) United States
(12) Patent Application Publication
(10) Pub. No.: US 2011/0293655 A1
(43) Pub. Date: Dec. 1, 2011

(54) Porcine Adenovirus 3-Based PRRSV Vaccines

(75) Inventor: Michael Sheppard, Phillip Island (AU)

(73) Assignee: IMUGENE LTD., Perth (AU)

(21) Appl. No.: 13/109,714

(22) Filed: May 17, 2011

Related U.S. Application Data
(60) Provisional application No. 61/348,925, filed on May 27, 2010.

(51) Int. Cl. A61K 39/12; A61P 31/14; A61P 37/04; C12N 7/01; C12N 15/63

(52) U.S. Cl. 424/199.1; 435/235.1; 435/320.1

(57) ABSTRACT

The invention relates to a porcine adenovirus 3 based vaccine for the treatment of PRRS virus infection where the PADV3 is a recombinant replication competent PADV3 that comprises a nucleic acid that encodes a novel fusion protein of PRRSV ORF6 and a modified PRRSV ORF5 either alone or in combination with a PRRSV ORF.

### RELATED APPLICATIONS

The present application claims benefit of priority of U.S. Provisional Application No. 61/348,925, which was filed on May 27, 2010 and is incorporated herein by reference in its entirety.

### FIELD OF THE INVENTION

The present invention relates to compositions and methods for the treatment of porcine reproductive and respiratory syndrome. More specifically, the compositions use expression constructs to encode specific subunits for eliciting an immune response in pigs.

### BACKGROUND OF THE INVENTION

Porcine reproductive and respiratory syndrome (PRRS) is one of the most economically important infectious diseases facing the swine industry. Infection with the PRRS virus is characterized by reproductive problems in sows (e.g. low farrowing rates, increased stillbirths) and respiratory problems in piglets. The PRRS virus has a particular affinity for the macrophages particularly the alveolar macrophages. Upon PRRS virus infection, the PRRS virus multiplies inside the alveolar macrophages resulting in destruction of up to 40% of macrophages of the animal. Once the macrophages are destroyed the pig's major defense mechanism is obliterated leaving the animal vulnerable to secondary infection by other bacterial and viral pathogens.

The PRRS virus is a well-characterized enveloped positive-stranded RNA virus. The genome of PRRSV is approximately 15 kb in length and consists of 9 open reading frames (ORFs). The four structural proteins of the virion GP3, 4, 5 and M are encoded by ORFs 3, 4, 5 and 6.

Even though there are only 4 structural proteins in the PRRS virion and there have many attempts to generate an effective vaccine to the PRRSV, studies are still ongoing. Individual proteins, co-expression of proteins and even multiple proteins have all been tried and in many different delivery systems including, purified protein, viral vectored and naked DNA. Nevertheless, despite extensive efforts in attempting to control PRRSV infections, the virus remains a significant burden on the swine industries across the world and the lack of a safe and effective vaccine remains a major barrier for controlling this disease.

### BRIEF SUMMARY OF THE INVENTION

The invention relates to a porcine adenovirus 3 based vaccine for the treatment of PRRS virus infection where the PADV3 is a recombinant replication competent PADV3 that comprises a nucleic acid that encodes a novel fusion protein of PRRSV ORF6 and a modified PRRSV ORF5 either alone or in combination with a PRRSV ORF.

More particularly, the present application provides description of a replication competent porcine adenovirus type 3 virus (PADV3) comprising a heterologous nucleic acid that encodes a fusion of PRRS virus ORF6 and ORF5, inserted into a non-essential site of the PADV3 wherein said ORF5 is a modified ORF5 that contains a spacer sequence to separate the neutralizing and non-neutralizing epitopes encoded by ORF5 wherein the sequence of the nucleic acid encoding the ORF6ORF5m is the sequence of SEQ ID NO:1 or SEQ ID NO:2, wherein the spacer sequence encodes a Pan DR T-helper cell epitope (PADRES) as encoded by a sequence GCTAAATTTGTCGCAGCCTGGACTCTTAAGGCAGCGGCT (SEQ ID NO:22) or the sequence of GCTAAATTTGTCGCAGCCTGGACTCTTAAGGCAGCGGCT (SEQ ID NO:22) in SEQ ID NO:1 or SEQ ID NO:2 is replaced by any other nucleic acid sequence that encodes a peptide of between 10 to 15 amino acids in length. It is contemplated that the non-essential site in the PADV3 is selected from the group consisting the E3 region, ORF 1-2 and 4-7 of E4, and the region between map units 97-99.5 of the PADV3 genome. More particularly, the non-essential site is the E3 region and said E3 region of said PADV3 is deleted and replaced with said nucleic acid that encodes the ORF6ORF5m. In other embodiments, it is contemplated that it may be appropriate to retain the E3 region and instead simply modify it to insert the heterologous nucleic acid.

In other embodiments, the non-essential site is the region between map units 97-99.5 of PADV3 genome and said nucleic acid that encodes the ORF6ORF5m is inserted into said region without deletion of the PADV3 map units 97-99.5. Alternatively, the region between map units 97-99.5 of PADV3 may be deleted and replaced with the heterologous nucleic acid.

In another aspect of the invention, in addition to ORF6ORF5m, the replication competent PADV3 further comprises a nucleic acid encoding PRRS ORF7. In such embodiments where the replication competent PADV3 comprises both ORF6ORF5m and ORF7, the ORF7 may be inserted into either the E3 region or the region between map units 97-99.5 of the porcine adenovirus 3 vector. For example, it is contemplated that the replication competent PADV3 contains the ORF7 in units 97-99.5 and the ORF6ORF5m in the E3 region. In another alternative, the replication competent PADV3 contains the ORF6ORF5m in units 97-99.5 and the ORF7 in the E3 region. In still another alternative, the replication competent PADV3 contains both the ORF7 and the ORF6ORF5m in the E3 region. In such an embodiment, the ORF7 may be under the control of the same promoter as the ORF6ORF5m and be expressed as a fusion protein with the ORF6ORF5m or it may be under the control of a separate promoter. In still another alternative, the replication competent PADV3 contains both the ORF7 and the ORF6ORF5m in the map units 97-99.5 of the PADV3 genome. Again, in such an embodiment, the ORF7 may be under the control of the same promoter as the ORF6ORF5m and be expressed as a fusion protein with the ORF6ORF5m or it may be under the control of a separate promoter. In any of these embodiments, the genome of the PADV3 may be deleted for one or more of the non-essential regions (including for example the E3 region or the region at map units 97-99.5 of the PADV3 genome) to create additional space for the insertion of the ORF7 and/or ORF6ORF5m.

In specific embodiments, the replication competent PADV3 may further comprising a nucleic acid that encodes another antigen for eliciting an immune response in pigs.

In particular embodiments, the ORF6ORF5m nucleic acid sequence encodes a fusion protein having the sequence of SEQ ID NO:3 or SEQ ID NO:4. In other embodiments the replication competent PADV3 comprises PRRS ORF7 encoded by a nucleic acid of SEQ. ID NO:18. In other embodiments, the ORF7 is encoded by SEQ ID NO:20.

Also contemplated herein is a composition comprising a first replication competent PADV3 as described herein, and a second recombinant expression vector that comprises an additional antigen for eliciting an immune response in pigs.

Another aspect of the invention relates to a vaccine for eliciting a protective response against PRRSV infection in pigs comprising a veterinarily acceptable vehicle or excipient and a replication competent PADV3 of the invention wherein the vaccine elicits neutralizing antibodies against PRRSV within two weeks of administration to a pig.

In particular embodiments, the vaccine may further comprise one or more additional antigen for vaccination of pigs wherein said additional one or more antigen is provided as a protein component in the veterinarily acceptable vehicle or excipient of said vaccine.

The invention also describes a vaccine for the protection of pigs against diseases caused by PRRSV, said vaccine comprising a recombinant PADV3 virus vector comprising a heterologous nucleic acid that encodes a fusion of PRRS virus ORF6 and ORF5, inserted into a non-essential site of the PADV3 wherein said ORF5 is a modified ORF5 that contains a spacer sequence to separate the neutralizing and non-neutralizing epitopes encoded by ORF5 wherein the sequence of the nucleic acid encoding the ORF6ORF5m is the sequence of SEQ ID NO:1 or SEQ ID NO:2, wherein the spacer sequence encodes a Pan DR T-helper cell epitope (PADRES) as encoded by a sequence GCTAAATTTGTCGCAGCCTGGACTCTTAAGGCAGCGGCT (SEQ ID NO:22) or the sequence of GCTAAATTTGTCGCAGCCTGGACTCTTAAGGCAGCGGCT (SEQ ID NO:22) in SEQ ID NO:1 or SEQ ID NO:2 is replaced by any other nucleic acid sequence that encodes a peptide of between 10 to 15 amino acids in length.

Such a vaccine may be further characterized in that the non-essential site is the E3 region and said E3 region of said PADV3 is deleted and replaced with said nucleic acid that encodes the ORF6ORF5m. In particular embodiments, the non-essential site is the region between map units 97-99.5 of PADV3 genome and said nucleic acid that encodes the ORF6ORF5m is inserted into said region without deletion of the PADV3 map units 97-99.5. In still other embodiments, the non-essential site is the region between map units 97-99.5 of the PADV3 genome and said region between map units 97-99.5 of the PADV3 genome is deleted and replaced with said nucleic acid that encodes the ORF6ORF5m. In still other embodiments, the PADV3 in the vaccine further comprises a nucleic acid encoding PRRS ORF7 inserted into either the E3 region or the region between map units 97-99.5 of the porcine adenovirus 3 vector.

In other embodiments, the non-essential site is the region between map units 97-99.5 of PADV3 genome and said nucleic acid that encodes the ORF6ORF5m is inserted into said region without deletion of the PADV3 map units 97-99.5. Alternatively, the region between map units 97-99.5 of PADV3 may be deleted and replaced with the heterologous nucleic acid.

In another aspect of the invention, in addition to ORF6ORF5m, the vaccine comprises PADV3 that encodes PRRS ORF7. In such embodiments where the replication competent PADV3 comprises both ORF6ORF5m and ORF7, the ORF7 may be inserted into either the E3 region or the region between map units 97-99.5 of the porcine adenovirus 3 vector. For example, it is contemplated that the replication competent PADV3 in the vaccine contains the ORF7 in units 97-99.5 and the ORF6ORF5m in the E3 region. In another alternative, the replication competent PADV3 in the vaccine contains the ORF6ORF5m in units 97-99.5 and the ORF7 in the E3 region. In still another alternative, the replication competent PADV3 in the vaccine contains both the ORF7 and the ORF6ORF5m in the E3 region. In such an embodiment, the ORF7 may be under the control of the same promoter as the ORF6ORF5m and be expressed as a fusion protein with the ORF6ORF5m or it may be under the control of a separate promoter. In still another alternative, the replication competent PADV3 in the vaccine contains both the ORF7 and the ORF6ORF5m in the map units 97-99.5 of the PADV3 genome. Again, in such an embodiment, the ORF7 may be under the control of the same promoter as the ORF6ORF5m and be expressed as a fusion protein with the ORF6ORF5m or it may be under the control of a separate promoter. In any of these embodiments, the genome of the PADV3 may be deleted for one or more of the non-essential regions (including for example the E3 region or the region at map units 97-99.5 of the PADV3 genome) to create additional space for the insertion of the ORF7 and/or ORF6ORF5m.

Also contemplated herein is a vaccine for eliciting a protective response against PRRSV infection in pigs comprising a composition that contains a PADV3 as described herein.

The vaccines described herein may be formulated for aerosol administration. In other embodiments, the vaccines are formulated for oral, nasal, intramuscular, subcutaneous, or intradermal delivery.

The invention further contemplates a method of immunizing a pig against PRRSV comprising administering to said pig a vaccine as described herein, wherein said immunization increases the presence of neutralizing antibodies against PRRSV in said pig within two weeks of the first administration of said vaccine to said pig. In specific embodiments, the administration is oral and/or nasal administration (e.g., via inhalation). In other embodiments, the administration is intramuscular administration.

Another aspect of the invention concerns an expression construct comprising a CMV promoter operatively linked to a nucleic acid that encodes an ORF6 fused to a modified ORF5 wherein the modified ORF5 has been modified to spatially separate the neutralizing and non-neutralizing epitopes, wherein said expression construct further comprises a nucleic acid that encodes PRRS ORF7 operatively linked to a major late promoter and said ORF5m encoding sequence and said ORF7 sequence comprise a polyA flanking sequence. More particularly, the expression construct comprises an ORF6 sequence has a nucleic acid sequence of SEQ ID NO:6, which is derived from the Lelystad strain of PRRS, SEQ ID NO:9 (a consensus sequence) or SEQ ID NO:15 (a ORF6 sequence from an asian strain of PRRS). In other specific embodiments, the expression construct comprises a modified ORF5 sequence that has a nucleic acid sequence of SEQ ID NO:14 (asian construct) or SEQ ID NO:11 (consensus). It should be noted that ORF5, may be an ORF5 from any PRRS strain as long as the ORF5 in the construct is modified as described herein and forms a fusion with an ORF 6. Likewise, the ORF6 may be from any strain of PRRS as long as it is expressed in the PADV3 expression construct as a single fusion protein with a modified ORF5. Exemplary ORF5 and ORF6 sequences are shown herein but the skilled person is aware of other such PRRS ORF5 and ORF6 sequences that may be readily modified for use in the vaccines and expression constructs described herein.

In further embodiments, the expression construct is characterized as a bicistronic construct in which a sequence of SEQ ID NO:17 encodes the ORF6OR5m fusion and a sequence of SEQ ID NO:20 encodes the ORF7. Also contemplated herein is a recombinant PADV3 that comprises such an expression construct.

### BRIEF DESCRIPTION OF SEVERAL VIEWS OF THE DRAWINGS

FIG. 1 shows an exemplary rPAV3 PRRS ORF6-5-7 expression construct of the invention in which the ORF7. The ORF6-5 sequence in this construct is encoded by a nucleic acid of SEQ ID NO:17 and the ORF7 insert has a sequence of SEQ ID NO:20.

FIG. 2 shows an exemplary study design time-line for the treatment of pigs using the vaccines of the present invention where the pigs are challenged with PRRSV strain VR-2332 isolate BIAH-001 at day 28 of the design.

FIG. 3 shows virus isolation, shown as percentage viremia, from the sera of pigs from three separate study groups: Group T1: mock vaccinated pigs (unvaccinated group); Group T2 vaccinated via intramuscular (IM) administration of the vaccine compositions of the invention and Group T3 vaccinated via oral/nasal administration of the vaccine compositions of the invention.

FIG. 4 shows individual pig lung scores to show the percentage total lung pathology in Groups T1 (unvaccinated), T2 (IM vaccination), and T3 (oral/nasal vaccination).

FIG. 5 shows average pig lung scores to show the percentage total lung pathology in Groups T1 (unvaccinated), T2 (IM vaccination), and T3 (oral/nasal vaccination).

### DETAILED DESCRIPTION OF THE INVENTION

After infection of swine with PRRSV there is a rapid rise of PRRSV-specific non-neutralizing antibodies while neutralizing antibodies are detectable not sooner than 3 weeks later. The problem with this is that while the animal raises an non-neutralizing antibody response against the structural proteins (GP3, 4, 5 and M) relatively rapidly, the lack of neutralizing antibodies production for 3 weeks results in ineffective vaccines as the neutralizing antibodies are needed sooner than 3 weeks after challenge. The present invention relies on the use of a specific expression construct in the preparation of a PRRS vaccine that produces an immediate generation of neutralizing antibodies in a pig in response to challenge with PRRSV.

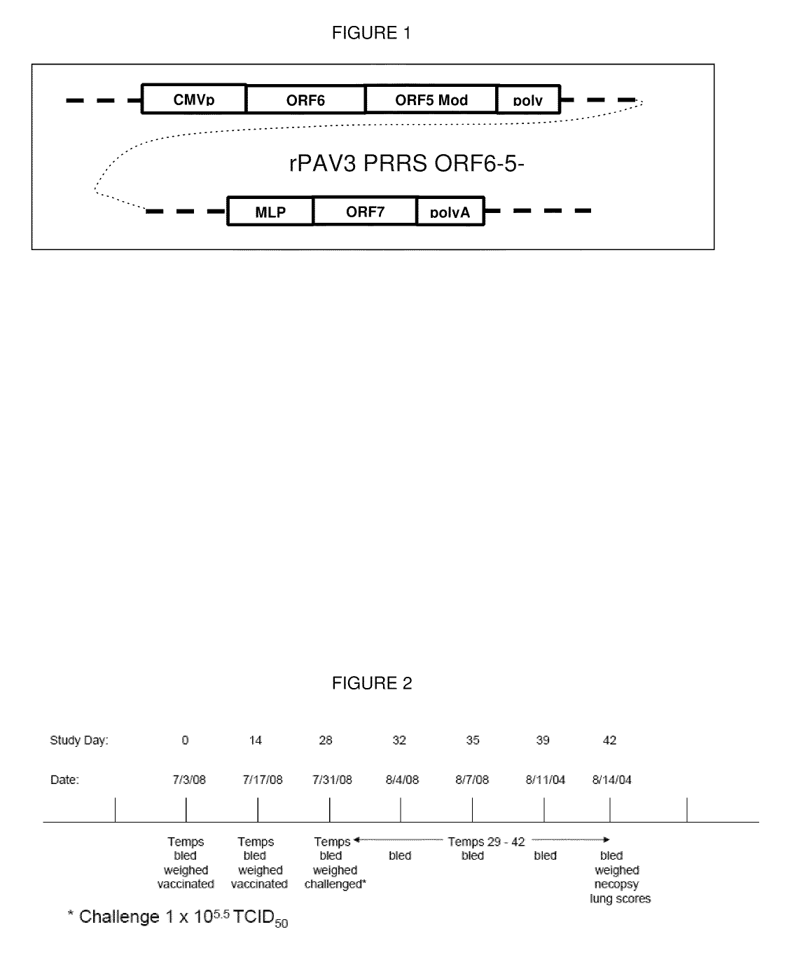

An expression construct is shown in FIG. 1. In this expression construct PRRSV ORF6 and a modified PRRSV ORF5 are expressed under the control of a CMV promoter as a single protein. The PRRSV ORF 6 sequence is a consensus sequence ORF6-encoding nucleic acid. The ORF5 is also a consensus ORF5 encoding nucleic acid but has been modified to include a Pan DR T-helper cell epitope (PADRE) between the neutralizing and the decoy epitope to minimize or eliminate the decoy effect of the non-neutralizing epitope. By way of explanation, at the N-terminus of GP5 protein encoded by ORF5 there are both non-neutralizing and neutralizing epitopes. In the North American strains these epitopes consist of amino acids 27 to 31 and 37 to 45 respectively. The non-neutralizing epitope (i.e., the epitope located at amino acids 27 to 31 of GP5) is highly immunodominant and exhibits features of a decoy epitope. It is possible that the decoy epitope of PRRSV GP5 is responsible for the reduced and delayed neutralizing antibody response. Therefore, in the present invention the decoy effect is minimized or eliminated by spatially separating the non-neutralizing and neutralizing epitopes by insertion of a PADRE sequences in between the two epitopes. The exemplary PADRE sequence used herein has a sequence of GCTAAATTTGTCGCAGCCTTGACTCTTAAGGCAGCGGCT (SEQ ID NO:22).

While a PADRE sequence is used to separate the neutralizing and non-neutralizing epitopes in exemplary embodiments, it should be understood that any linker may be used to increase the space between these two epitopes expressed by ORF5 and as such instead of the PADRE sequence the epitopes may simply be separated by a peptide linker which may be of any length preferably it is 6 amino acids in length, 11 amino acids in length, 16 amino acids in length or 20 amino acids in length. Other embodiments contemplate linkers that are 6 to 11 amino acids in length, 11 to 16 amino acids in length, 16 to 20 amino acids in length, 16 to 25 amino acids in length or 20 to 30 amino acids in length. Specific embodiments contemplate a linker that is a glycine succinate linker, an amino acid linker or combination thereof. For example, the linker may be one that is Gly(SerGlyGly)2SerGly, (SEQ ID NO. 24), or other variants thereof.

In specific embodiments, the ORF5 is an ORF5 from an Asian strain of PRRSV and comprises a sequence of:

ATGTTGGGGAAATGCTTGACCGCGGGCTGTTGCTCGCAATTGCTTTTTTTGTGGTGTATCGTGCCGT TCTTTTCTGCTGTGCCCGCCAGCGCCAACAGAAACAACAGCTCCCATTTACAGCTGATTTACAACTTG ACGCTATGTGAGCTGAATGGCACAGATTGGCTAGCTGGCAAATTTGATTGGGCAGTGGAGAGTTTTG TCATCTTTCCCGTTTTGACTCACATTGTCTCCTATGGTGCCCTCACTACTAGCCATTTCCTTGACACA GCCGCTTTAGTCACTGTGTCTACCGCCGGTTTTCTTCACGGGCGGTATGTCCTAAGCAGCATCTACG CTGTCTGTGCCCTGGCTGCGTTGACTTGCTTCGTCATTAGGTTTGCAAAGAATTGCATGTCCTGGCG CTACGCGTGCACCAGATACACCAACTTTCTTCTGGACACTAAGGGCAGACTCTATCGGTGGCGGTC GCCTGTCATCATAGAGAGAAGGGGCAAAGTTGAGGTCGAAGGTCATCTGATCGACCTCAAAAGAGTT GTGCTTGATGGTTCCGTGGCAACCCCTGTAACCAGAGTTTCAGCGGAACAATGGGGTCGTCCTTAG SEQ ID NO: ______, wherein that sequence encodes a protein having the sequence of M L G K C L T A G C C S Q L L F L W C I V P F F S A V P A S A N R N N S S H L Q L I Y N L T L C E L N G T D W L A G K F D W A V E S F V I F P V L T H I V S Y G A L T T S H F L D T A A L V T V S T A G F L H G R Y V L S S I Y A V C A L A A L T C F V I R F A K N C M S W R Y A C T R Y T N F L L D T K G R L Y R W R S P V I I E R R G K V E V E G H L I D L K R V V L D G S V A T P V T R V S A E Q W G R P (SEQ ID NO: ______).

The expression cassette shown in FIG. 1 is inserted into a replication competent PADV3 vector in which the sequence of SEQ ID NO:1 or SEQ ID NO:2 is used to replace the E3 region of the PADV3 genome or alternatively, the sequence of SEQ ID NO:1 or SEQ ID NO:2 is used to replace the region that forms map units 97 to 99.5 of PADV3. It should be understood that the sequences of SEQ ID NO:1 or SEQ ID NO:2 may be inserted into the E3 or map units 97 to 99.5 of PADV3 or alternatively, the E3 and/or the map units 97 to 99.5 are deleted and replaced with the sequences of SEQ ID NO:1 or SEQ ID NO:2.

In specific embodiments, the expression cassette further comprises a nucleic acid that encodes PRRSV ORF7 under the control of a major late promoter (SEQ ID NO:4).

The porcine adenovirus (PADV) expression system is an attractive candidate for the production of a PRRS vaccine. Porcine adenoviruses are able to replicate efficiently to high titers; provide cloning space; PADV permit the expression of recombinant proteins in many porcine cell lines and tissues; express multiple genes in the same cell line or tissue; accurately express and modify the recombinant protein. While insertion into the E3 region (preferably where the E3 region has been deleted) or the region at map units 97 to 99.5 of PADV3 is preferred, it should be understood that the expression cassette having a sequence of SEQ ID NO:1 or SEQ ID NO2 may be inserted anywhere in the PADV3 genome. As such, it is contemplated that these sequences may be inserted into non-essential sequence of PADV-3 selected from the group consisting of the E3 region, ORF 1-2 and 4-7 of E4, the region between the end of E4 and the ITR of the porcine adenovirus 3 genome.

The invention contemplates a composition comprising a first recombinant expression vector as described above and a second recombinant expression vector that comprises an additional antigen for eliciting an immune response in pigs. Also contemplated are vaccines for eliciting a protective response against PRRSV infection in pigs comprising such a composition.

Other aspects of the invention relate to a vaccine for eliciting a protective response against PRRSV infection in pigs comprising a veterinarily acceptable vehicle or excipient and a recombinant expression vector comprising a nucleic acid sequence that encodes a fusion protein comprising ORF6 linked to a modified ORF5, wherein the ORF5 is modified to contain a PADRE epitope wherein the nucleic acid encoding the fusion protein is operably linked to a promoter, and wherein the ORF6 sequence is at the amino terminal of the modified ORF5-encoded. In some embodiments, the vaccine may advantageously further comprise one or more additional antigen for vaccination of pigs wherein said additional one or more antigen is provided as a protein component in the veterinarily acceptable vehicle or excipient of said vaccine. An exemplary PADRE sequence is shown in SEQ ID NO:23 and encoded by a nucleic acid of SEQ ID NO:22.

The invention specifically contemplates preparation and use of a vaccine for the protection of pigs against PRRSV, said vaccine comprising a recombinant virus vector comprising a promoter operably linked to a sequence that encodes a fusion protein of ORF6-linker-modified ORF5, wherein the modified ORF5 comprises a PADRE epitope separating the neutralizing epitope from the non-neutralizing epitope of the GP5 protein encoded by the modified ORF5. Specifically, the recombinant vector contains an expression cassette have a sequence of SEQ ID NO:1 or SEQ ID NO:2 wherein said nucleic acid encode the ORF6-linker-modified ORF5 fusion protein. In specific embodiments, the vector further comprises a nucleic acid that encodes ORF7. More particularly, the ORF7 is under the control of an MLP promoter and comprises a sequence of SEQ ID NO:20.

The vaccines may be formulated for any route of administration including for example oral, nasal, intramuscular, subcutaneous, or intradermal delivery. In preferred embodiments, the vaccine is formulated for aerosol administration.

The invention also contemplates a method for eliciting an immune response in a porcine subject comprising administering vaccines of the invention to the porcine subject in an amount effective to elicit a protective immune response in said porcine subject such that one or more of the symptoms of PRRS infection in the heard is eliminated or avoided.

The present invention relies on conventional techniques for the construction of improved viral vaccines of the invention for the treatment of pigs. The viral vaccines may be constructed from any viral vector that can be used to infect pigs and may include vectors such as but not limited to an adenoviral vector, an adenoassociated viral vector, a lentiviral vector, a herpes viral vector, a pox viral vectors. In exemplary embodiments, the viral vectors are porcine adenoviral vectors. Vaccines made with porcine viral vectors are known to those of skill in the art (see e.g., U.S. Pat. Nos. 7,323,177; 7,297,537; 6,852,705).

The present invention relates to methods of preparing and use of recombinant PADV3-based viral vaccine compositions that can be administered to a population of pigs for protective immunity against PRRSV infection.

The existing vaccines do not meet the long-felt need in the art for an effective vaccine against diseases caused by PRRSV. To combat the problems with the existing treatments PRRSV infection, the present inventors have developed a new vaccine for conferring protective immunity to pigs. The vaccine is based on a PADV3 viral expression system that affords expression of a specific fusion protein of ORF6 and modified ORF5 in a subunit vaccine. The fusion protein that forms the antigen in the PADV3 based viral vaccine elicits a response based on neutralizing and non-neutralizing antibodies in a matter of days thereby providing a significant advantage over the methods available in the prior which are ineffective due to lack of production of neutralizing antibodies in a therapeutically relevant time frame as the production of the neutralizing antibodies in those previous methods was not elicited within the first three weeks of infection. These features and methods and compositions for using recombinant viral vaccines for PRRSV disease are described in further detail herein below.

In general terms the vaccine of the present invention is comprised of a viral expression vector that is made of a PADV3 viral genome. Porcine adenoviruses are well known to those of skill in the art and have been extensively characterized. In specific embodiments, the porcine adenovirus 3 used as the vector in the methods and compositions described herein is one that is deleted for E3. In other embodiments, the PADV3 may be further deleted for other non-essential regions. Given the teaching provided herein however, the skilled person may use any virus that infects pigs to prepare vaccines of the invention.

In the vaccines prepared herein the promoter used may be any promoter that can drive expression of a heterologous gene of interest in an viral construct. Such promoters include but are not limited to avian adenoviral major late promoter (MLP), CMVp, PGK-, E1-, SV40 early promoter (SVG2), SV40 late promoter, SV-40 immediate early promoter, T4 late promoter, and HSV-I TK (herpesvirus type 1 thymidine kinase) gene promoter, the RSV (Rous Sarcoma Virus) LTR (long terminal repeat) and the PGK (phosphoglycerate kinase) gene promoter. Many other mammalian promoters known to those of skill in the art also may be used.

The promoter used in the vaccines described herein drives the expression of an in-frame fusion of a PRRSV ORF6 linked via a short peptide linker to a modified ORF5-encoding nucleic acid sequence wherein the modification is an inclusion of a nucleic acid sequence that encodes a PADRE epitope in between the neutralizing and non-neutralizing epitopes of ORF5. The exemplary promoter used is the CMV promoter. In some embodiments, the MLP promoter is used. In a specific example the vaccines further comprising PRRSV ORF7 wherein the ORF7 is operatively linked to a MLP promoter and having a polyA tail (SEQ ID NO:20 shows the nucleic acid sequence of the expression construct for ORF7, the sequence of SEQ ID NO:18 shows the ORF7 nucleic acid and SEQ ID NO:19 shows the amino acid of ORF7 used in an exemplary embodiment).

The expression constructs used herein will preferably comprise DNA that encodes the protein to be delivered. Such DNA may be comprised of the nucleotide bases A, T, C, and G, but also may include any analogs or modified forms of such bases. Such analogs and modified bases are well known to those of skill in the art, and include but are not limited to methylated nucleotides, internucleotide modifications such as uncharged linkages and thioates, use of sugar analogs, and modified and/or alternative backbone structures, such as polyamides.

In exemplary embodiments, the viral vectors are porcine adenovirus vectors 3 vectors that are preferably replication-competent but may be replication-defective in a target cell. In the event that the vectors are replication-defective, the vectors may require use of a helper cell or a helper virus to facilitate replication. Use of helper cells or helper viruses to promote replication of replication-defective adenoviral vectors is routine and well-known in the art. Typically, such helper cells provide the function of the entity that has been knocked out of the recombinant adenoviral vector to render it replication defective.

A replication competent vector on the other hand may be referred to as a “helper-free virus vector” in that it does not require a second virus or a cell line to supply something defective in the vector. The preparation of viral vector-based vaccines that contain the fusion protein encoded by the sequence of SEQ ID NO:1 or SEQ ID NO:2 is limited only by the insertion capacity of the given viral genome and ability of the recombinant viral vector to express the inserted heterologous sequences. For example, where the vector is an adenoviral vector, adenovirus genomes can accept inserts that increase the size of the recombinant adenovirus to at least 105% of the wild-type genome length and remain capable of being packaged into virus particles. The insertion capacity of such viral vectors can be increased by deletion of non-essential regions and/or deletion of essential regions, such as, for example, El function, whose function can then be provided by a helper cell line, such as one providing El function. In some embodiments, a heterologous polynucleotide encoding the protein of interest (in this case the PCV2 ORF2 and/or any additional therapeutic protein that is to be used in the vaccine) is inserted into an adenovirus E3 gene region. In other embodiments, the non-essential portions of the E3 region are deleted and the heterologous polynucleotide encoding the PRRSV ORF6ORF5m and/or the PRRSV ORF7 protein(s) of interest is inserted at that gap left by the deletion. In some preferred embodiments, where the recombinant adenoviral vector is a porcine adenovirus serotype 3 (PAdV-3) based adenoviral vector, in which the expression construct containing the PRRSV ORF6ORF5m and/or the PRRSV ORF7 protein(s) encoding nucleic acid (and/or other nucleic acid) is inserted into the region of the PADV-3 genome located after the polyadenylation signal for PAdV-3 E3 and before the start of the ORF for the PAdV-3 fibre gene.

In some embodiments, an adenovirus is created where the insertion or the deletion followed by the insertion is in the El gene region of the adenovirus the vector is then propagated in a helper cell line providing El function. Other regions of PADV-3 into which the heterologous gene may be inserted include the E4 region. Where the recombinant adenoviral vector is a PADV-3 based vector, the entire E4 region, except that region that encodes PADV ORF3 can be deleted to make room for the heterologous gene. For example, the region at map units 97-99.5 is a particularly useful site for insertion of the heterologous gene. As shown in Li et al. (Virus Research 104 181-190 (2004)), the PADV-3 E4 region located at the right-hand end of the genome is transcribed in a leftward direction and has the potential to encode seven (pl-p7) ORFs. Of these only ORF p3 is essential for the replication. As such, much if not all of the rest of the E4 region may readily be deleted without rendering the virus replication defective, thereby allowing for more room for heterologous inserts. In one embodiment of the invention, insertion can be achieved by constructing a plasmid containing the region of the adenoviral genome into which insertion of the polynucleotide encoding for a desired therapeutic protein is desired. The plasmid is then digested with a restriction enzyme having a recognition sequence in that adenoviral portion of the plasmid, and a heterologous polynucleotide sequence is inserted at the site of restriction digestion. The plasmid, containing a portion of the adenoviral genome with an inserted heterologous sequence, is co-transformed, along with an adenoviral genome or a linearized plasmid containing the adenoviral genome into a bacterial cell (such as, for example, E. coli). Homologous recombination between the plasmids generates a recombinant adenoviral genome containing inserted heterologous sequences. In these embodiments, the adenoviral genome can be a full-length genome or can contain one or more deletions as discussed herein.

Deletion of adenoviral sequences, for example to provide a site for insertion of heterologous sequences or to provide additional capacity for insertion at a different site, can be accomplished by methods well-known to those of skill in the art. For example, for adenoviral sequences cloned in a plasmid, digestion with one or more restriction enzymes (with at least one recognition sequence in the adenoviral insert) followed by ligation will, in some cases, result in deletion of sequences between the restriction enzyme recognition sites. Alternatively, digestion at a single restriction enzyme recognition site within the adenoviral insert, followed by exonuclease treatment, followed by ligation will result in deletion of adenoviral sequences adjacent to the restriction site. A plasmid containing one or more portions of the adenoviral genome with one or more deletions, constructed as described above, can be co-transfected into a bacterial cell along with an adenoviral genome (full-length or deleted) or a plasmid containing either a full-length or a deleted genome to generate, by homologous recombination, a plasmid containing a recombinant genome with a deletion at one or more specific sites. Adenoviral virions containing the deletion can then be obtained by transfection of mammalian cells including but not limited to the stably transformed cells containing the additional fibre gene described herein, with the plasmid containing the recombinant adenoviral genome. The insertion sites may be adjacent to and transcriptionally downstream of endogenous promoters in the adenovirus. An “endogenous” promoter, enhancer, or control region is native to or derived from adenovirus. Restriction enzyme recognition sequences downstream of given promoters that can be used as insertion sites, can be easily determined by one of skill in the art from knowledge of part or all of the sequence of adenoviral genome into which the insertion is desired. Alternatively, various in vitro techniques are available to allow for insertion of a restriction enzyme recognition sequence at a particular site, or for insertion of heterologous sequences at a site that does not contain a restriction enzyme recognition sequence. Such methods include, but are not limited to, oligonucleotide-mediated heteroduplex formation for insertion of one or more restriction enzyme recognition sequences (see, for example, Zoller et al. (1982) Nucleic Acids Res. 10:6487-6500; Brennan et al. (1990) Roux's Arch. Dev. Biol. 199:89-96; and Kunkel et al. (1987) Meth. Enzymology 154:367-382) and PCR-mediated methods for insertion of longer sequences. See, for example, Zheng et al. (1994) Virus Research 31:163-186.

Expression of a heterologous sequence inserted at a site that is not downstream from an endogenous promoter also can be achieved by providing, with the heterologous sequence, a transcriptional regulatory sequences that are active in eukaryotic cells. Such transcriptional regulatory sequences can include cellular promoters such as, for example (DHFR promoter), the viral promoters such as, for example, herpesvirus, adenovirus and papovavirus promoters and DNA copies of retroviral long terminal repeat (LTR) sequences. In such embodiments, the heterologous gene is introduced in an expression construct in which the heterologous gene is operatively linked to such transcriptional regulatory elements.

In specific exemplary embodiments, the fusion protein encoded by the ORF6ORF5m nucleic acid is placed under the control of a promoter, such as for example, the CMV promoter in order to provide constitutive transcription. In a PADV3-based viral vector, continued translation of the recombinant ORF6ORF5m mRNA can be achieved by placing the nucleic acid encoding the ORF6ORF5m expression construct downstream of the PADV-3 MLP/TPL sequence. It should be understood that preparation of the recombinant adenoviral vectors includes propagation of the cloned adenoviral genome as a plasmid and rescue of the infectious virus from plasmid-containing cells.

The presence of viral nucleic acids can be detected by techniques known to one of skill in the art including, but not limited to, hybridization assays, polymerase chain reaction, and other types of amplification reactions. Similarly, methods for detection of proteins are well-known to those of skill in the art and include, but are not limited to, various types of immunoassay, ELISA, Western blotting, enzymatic assay, immunohistochemistry, etc. Diagnostic kits comprising the nucleotide sequences of the invention may also contain reagents for cell disruption and nucleic acid purification, as well as buffers and solvents for the formation, selection and detection of hybrids. Diagnostic kits comprising the polypeptides or amino acid sequences of the invention may also comprise reagents for protein isolation and for the formation, isolation, purification and/or detection of immune complexes.

In addition to the PRRSV ORF6ORF5m and ORF7 nucleic acids, other exogenous (i.e., foreign) nucleotide sequences can be incorporated into the adenovirus. These other exogenous sequences can consist of one or more gene(s) of interest or other nucleotide sequences that are not genes but have other functions of therapeutic interest. In the context of the present invention, a nucleotide sequence or gene of interest can code either for an antisense RNA, short hairpin RNA, a ribozyme or for an mRNA which will then be translated into a protein of interest. Such a nucleotide sequence or gene may comprise genomic DNA, complementary DNA (cDNA) or of mixed type (minigene, in which at least one intron is deleted). The nucleotide sequence or gene can encode a regulatory or therapeutic function, a mature protein, a precursor of a mature protein, in particular a precursor that comprises a signal peptide, a chimeric protein originating from the fusion of sequences of diverse origins, or a mutant of a natural protein displaying improved or modified biological properties. Such a mutant may be obtained by, deletion, substitution and/or addition of one or more nucleotide(s) of the gene coding for the natural protein, or any other type of change in the sequence encoding the natural protein, such as, for example, transposition or inversion.

The gene that is being delivered by the vector may be placed under the control of elements (DNA control sequences) suitable for its expression in a host cell. Suitable DNA control sequences are understood to mean the set of elements needed for transcription of a gene into RNA (antisense RNA or mRNA) and for the translation of an mRNA into protein. For example, these elements would include at least a promoter. The promoter may be a constitutive promoter or a regulatable promoter, and can be isolated from any gene of eukaryotic, prokaryotic or viral origin, and even adenoviral origin. Alternatively, it can be the natural promoter of the gene of interest. Generally speaking, a promoter used in the present invention may be modified so as to contain regulatory sequences. Exemplary promoters may include tissue specific promoters when the gene is to be targeted to a given tissue type. Other conventional promoters that may be used include but are not limited to the HSV-I TK (herpesvirus type 1 thymidine kinase) gene promoter, the adenoviral MLP (major late promoter), the RSV (Rous Sarcoma Virus) LTR (long terminal repeat), the CMV immediate early promoter, SV-40 immediate early promoter, and the PGK (phosphoglycerate kinase) gene promoter, for example, permitting expression in a large number of cell types.

The viral vectors or indeed a pharmaceutical composition comprising the viral vectors can additionally include at least one immunogen from at least one additional pig pathogen, e.g.: an additional Porcine Reproductive and Respiratory Syndrome (PRRS) antigen, Mycoplasma hyopneumoniae, Actinobacillus pleuropneumoniae, E. coli, Bordetella bronchiseptica, Pasteurella multocida, Erysipelothrix rhusiopathiae, Pseudorabies, Hog cholera, Swine Influenza, and Porcine Parvovirus (PPV). Thus, vector-based compositions can include at least one immunogen from at least one additional pig pathogen, such as a vector expressing a sequence from this pathogen, wherein the vector is also capable of expressing the ORF6ORF5m and/or ORF7 described above. Alternatively, the vaccine composition can be made of one vector component that expresses the ORF6ORF5m and ORF7 as described herein and a second component that can either be a recombinant vector expressing a second immunogen or the second component is a composition that contains the isolated immunogen that has been isolated from another source.

It should be understood that while in some circumstances it might be desirable to incorporate the whole gene into the vector, other vectors can be constructed that comprise only a portion of the nucleotide sequences of genes can be used (where these are sufficient to generate a protective immune response or a specific biological effect) rather than the complete sequence as found in the wild-type organism. Where the genes contain a large number of introns, a cDNA may be preferred.

As noted above, the gene may be inserted under the control of a suitable promoter. In addition the vector also may comprise enhancer elements and polyadenylation sequences. Promoters and polyadenylation sequences which provide successful expression of foreign genes in mammalian cells and construction of expression cassettes, are known in the art, for example in U.S. Pat. No. 5,151,267, the disclosures of which are incorporated herein by reference.

The term “expression cassette” refers to a natural or recombinantly produced nucleic acid molecule that is capable of expressing a gene or genetic sequence in a cell. An expression cassette typically includes a promoter (allowing transcription initiation), and a sequence encoding one or more proteins or RNAs. Optionally, the expression cassette may include transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences. Optionally, the expression cassette may include a gene or partial gene sequence that is not translated into a protein. The nucleic acid can effect a change in the DNA or RNA sequence of the target cell. This can be achieved by hybridization, multi-strand nucleic acid formation, homologous recombination, gene conversion, RNA interference or other yet to be described mechanisms.

The viral vectors may comprise more than one foreign gene. The methods of the invention are preferably used to provide protection against PRRSV-based disease in pigs. While exemplary embodiments of the present invention are such that the heterologous nucleotide (also referred to herein in as heterologous nucleic acid) is one which encodes a protein, it should be understood that the heterologous nucleotide may in fact be any polynucleotide containing a sequence whose presence or transcription in a cell is desired. Thus the vectors may be used to deliver any polynucleotide that, for example, causes sequence-specific degradation or inhibition of the function, transcription, or translation of a gene.

The immunogen compositions other than the PRRSV ORF6ORF5m and/or PRRSV ORF7 can be recombinantly produced or extracted from natural sources or may be chemically synthesized. For example, the immunogen compositions other than the PRRSV ORF6ORF5m, can be isolated and/or purified from infected or transfected cells; for instance, to prepare compositions for administration to pigs; however, in certain instances, it may be advantageous not to isolate and/or purify an expression product from a cell; for instance, when the cell or portions thereof enhance the immunogenic effect of the polypeptide. Protein purification and/or isolation techniques used to achieve this are well known to those of skill in the art and in general, can include: precipitation by taking advantage of the solubility of the protein of interest at varying salt concentrations, precipitation with organic solvents, polymers and other materials, affinity precipitation and selective denaturation; column chromatography, including high performance liquid chromatography (HPLC), ion-exchange, affinity, immunoaffinity or dye-ligand chromatography; immunoprecipitation, gel filtration, electrophoretic methods, ultrafiltration and isoelectric focusing, and their combinations.

It has previously been shown that a modified rPAdV-gp55 grown in PK-15 cells when administered to commercially available Large White Pigs by sub-cutaneous or oral routes completely protected pigs from lethal challenge with CSFV when given as subcutaneous injection or by the oral route. In the context of the present invention a similar approach may be taken to administer the vaccines or recombinant PADV3 virus comprising the PRRSV ORF6ORF5m either alone or in combination with gp55 or some other antigen to confer an effective immunity or vaccination of the pigs against disease.

Specifically contemplated herein are pharmaceutical compositions comprising a therapeutically effective amount of a recombinant adenovirus vector, recombinant adenovirus or recombinant protein, prepared according to the methods of the invention, in combination with a pharmaceutically acceptable vehicle and/or an adjuvant. Such a pharmaceutical composition can be prepared and dosages determined according to techniques that are well-known in the art. The pharmaceutical compositions of the invention can be administered by any known administration route including, but not limited to, systemically (for example, intravenously, intratracheally, intravascularly, intrapulmonarilly, intraperitoneally, intranasally, parenterally, enterically, intramuscularly, subcutaneously, intratumorally or intracranially), by oral administration, by aerosolization or intrapulmonary instillation. Administration can take place in a single dose or in doses repeated one or more times after certain time intervals. The appropriate administration route and dosage will vary in accordance with the situation (for example, the individual being treated, the disorder to be treated or the gene or polypeptide of interest), but can be determined by one of skill in the art.

In specific embodiments, female pigs will be inoculated with a viral vector composition that comprises a nucleic acid that expresses at least the PRRS ORF6ORF5m (as exemplified by the sequences set forth in SEQ ID NO:1 or SEQ ID NO:2) either alone or in combination with PRRS ORF7 (as exemplified by the sequence set forth in SEQ ID NO:4). The animal may be inoculated prior to breeding; and/or prior to serving, and/or during gestation (or pregnancy); and/or prior to the perinatal period or farrowing; and/or repeatedly over a lifetime, to prevent myocarditis and/or abortion and/or intrauterine infection associated with PRRS, and other pathologic sequelae associated with PRRSV infection; or, to elicit an immunogenic or protective response against PRRSV and thereby prevent any disease or symptom associated with PRRSV infection. Such symptoms and diseases include but are not limited to enzootic pneumonia in grower/finisher units, loss of appetite, elevated body temperature to 39-40° C., abortion and in particular later term abortion, transient discoloration of ears, sows early farrowing, prolonged anoestrus and delayed returns to heat post-weaning, coughing and other respiratory symptoms, reluctance to drink in farrowing sows, agalactia and or mastitis in farrowing sows, discoloration of the skin and pressure sores associated with small vesicles in farrowing sows, mummified piglets, increased stillbirths, clinical pneumonia. The acute phase of PRRSV infection in a herd lasts up to 6 weeks, and is characterised by early farrowings, increases in stillbirths, weak pigs and an increase in the numbers of large mummified pigs that have died in the last three weeks of pregnancy. In some herds, these may reach up to 30% of the total pigs born. Piglet mortality peaks at 70% in weeks 3 or 4 after the onset of symptoms and only returns to pre-infected levels after 8-12 weeks. The reproductive problems often persist for 4-8 months in the herd. The PRRSV infection in the long term shows symptoms such as acute extensive consolidating pneumonia, formation of multiple abscesses, persistent diarrhea, pale skin, coughing, sneezing, discharge from eyes, increased respiratory rates, mortality of 15%, overall poor and stunted growth. The symptoms of the disease become evident within 1-3 weeks of weaning.

In another embodiment, piglets are inoculated within the first weeks of life, e.g., inoculation at one and/or two and/or three and/or four and/or five weeks of life. More preferably, piglets are first inoculated within the first week of life or within the third week of life (e.g., at the time of weaning). Even more advantageous, such piglets are then boosted two (2) to four (4) weeks later (after being first inoculated). The piglets may be from vaccinated or unvaccinated females. Thus, both offspring, as well as female pig can be administered the compositions of the invention in order to increase the life expectancy of the piglets and their mothers.

The invention further provides for methods of treatment in which a therapeutically effective amount of a recombinant adenoviral vector (e.g., a PADV-3 adenoviral vector) that contains ORF6ORF5m fusion protein as the therapeutic antigen.

The antigens other than the fusion protein product of ORF6ORF5m nucleic acids (as exemplified by SEQ ID NO:1 or SEQ ID NO:2) that are used in combination with the modified fusion protein product can be either native or recombinant antigenic polypeptides or fragments. They can be partial sequences, full-length sequences, or even fusions (e.g., having appropriate leader sequences for the recombinant host, or with an additional antigen sequence for another pathogen). The preferred antigenic polypeptide to be expressed by the virus systems of the present invention contain full-length (or near full-length) sequences encoding antigens. Alternatively, shorter sequences that are antigenic (i.e., encode one or more epitopes) can be used. The shorter sequence can encode a “neutralizing epitope,” which is defined as an epitope capable of eliciting antibodies that neutralize virus infectivity in an in vitro assay. Preferably the peptide should encode a “protective epitope” that is capable of raising in the host a “protective immune response;” i.e., an antibody- and/or a cell-mediated immune response that protects an immunized host from infection.

In addition, any of the vaccines in the present invention also may comprise an adjuvant. An “adjuvant” is any substance added to a vaccine to increase the immunogenicity of the vaccine. The use of adjuvants in vaccine compositions are well known in the art: for example, bovine serum albumin (BSA), human serum albumin (HSA) and keyhole limpet hemocyanin (KLH). Some adjuvants are believed to enhance the immune response by slowly releasing the antigen, while other adjuvants are strongly immunogenic in their own right and are believed to function synergistically. Known vaccine adjuvants include, but are not limited to, oil and water emulsions (for example, complete Freund's adjuvant and incomplete Freund's adjuvant), Corynebacterium parvum, Bacillus Calmette Guerin, aluminum hydroxide, glucan, dextran sulfate, iron oxide, sodium alginate, Bacto-Adjuvant, certain synthetic polymers such as poly amino acids and co-polymers of amino acids, saponin, “REGRESSIN” (Vetrepharm, Athens, Ga.), “AVRIDINE” (N,N-dioctadecyl-N′,N′-bis(2-hydroxyethyl)-propanediamine), paraffin oil, muramyl dipeptide and the like. In certain embodiments, tocopherol may be used as an adjuvant.

Genes for desired antigens or coding sequences thereof which can be inserted include those of organisms which cause disease in mammals, particularly bovine pathogens such as foot-and-mouth disease virus, bovine rotavirus, bovine coronavirus, bovine herpes virus type 1, bovine respiratory syncytial virus, bovine parainfluenza virus type 3 (BPI-3), bovine diarrhea virus, Pasteurella haemolytica, Haemophilus somnus and the like. Genes encoding antigens of human pathogens also may be useful in the practice of the invention. The vaccines of the invention carrying foreign genes or fragments can also be orally administered in a suitable oral carrier, such as in an enteric-coated dosage form. Oral formulations include such normally-employed excipients as, for example, pharmaceutical grades of mannitol, lactose, starch, magnesium stearate, sodium saccharin cellulose, magnesium carbonate, and the like. Oral vaccine compositions may be taken in the form of solutions, suspensions, tablets, pills, capsules, sustained release formulations, or powders, containing from about 10% to about 95% of the active ingredient, preferably about 25% to about 70%. Oral and/or intranasal vaccination may be preferable to raise mucosal immunity (which plays an important role in protection against pathogens infecting the respiratory and gastrointestinal tracts) in combination with systemic immunity.

In addition, the vaccine can be formulated into a suppository. For suppositories, the vaccine composition will include traditional binders and carriers, such as polyalkaline glycols or triglycerides. Such suppositories may be formed from mixtures containing the active ingredient in the range of about 0.5% to about 10% (w/w), preferably about 1% to about 2%.

Protocols for administering to animals the vaccine composition(s) of the present invention are within the skill of the art in view of the present disclosure. Those skilled in the art will select a concentration of the vaccine composition in a dose effective to elicit an antibody and/or T-cell mediated immune response to the antigenic fragment or another type of therapeutic or prophylactic effect. Within wide limits, the dosage is not believed to be critical. The timing of administration may also be important. For example, a primary inoculation preferably may be followed by subsequent booster inoculations if needed. It may also be preferred, although optional, to administer a second, booster immunization to the animal several weeks to several months after the initial immunization. To insure sustained high levels of protection against disease, it may be helpful to readminister a booster immunization to the animals at regular intervals, for example once every several years. Alternatively, an initial dose may be administered orally followed by later inoculations, or vice versa. Preferred vaccination protocols can be established through routine vaccination protocol experiments.

The dosage for all routes of administration of in vivo recombinant virus vaccine depends on various factors including, the size of host/patient, nature of infection against which protection is needed, carrier and the like and can readily be determined by those of skill in the art. By way of non-limiting example, a dosage of between 10² pfu and 10¹⁵ pfu, preferably between 10⁴ and 10¹³ pfu, more preferably between 10⁵ to 10¹¹ pfu and the like can be used. As with in vitro subunit vaccines, additional dosages can be given as determined by the clinical factors involved.

The invention also includes a method for providing gene delivery to a mammal, and particularly to pigs, to control a gene deficiency, to provide a therapeutic gene or nucleotide sequence and/or to induce or correct a gene mutation. The method can be used, for example, in the treatment of conditions including, but not limited to hereditary disease, infectious disease, cardiovascular disease, and viral infection. These kinds of techniques are currently being used by those of skill in the art for the treatment of a variety of disease conditions. Examples of foreign genes, nucleotide sequences or portions thereof that can be incorporated for use in a conventional gene therapy include, cystic fibrosis transmembrane conductance regulator gene, human minidystrophin gene, alpha-1-antitrypsin gene, genes involved in cardiovascular disease, and the like.

For the purposes of the present invention, the vectors, cells and viral particles prepared by the methods of the invention may be introduced into a subject either ex vivo, (i.e., in a cell or cells removed from the patient) or directly in vivo into the body to be treated.

### EXAMPLES

### Example 1

### PAV3 Recombinant Viruses—Generation, Growth and Titration

In order to construct the exemplary vaccines of the present invention the gene of interest, e.g., the ORF6, ORF5m, ORF7 of PRRS is PCR amplified with appropriate PCR primers to introduce new cloning restriction sites therein. The PCR product is then cloned into plasmid pCR-2.1 TOPO vector or any other suitable vector using standard cloning procedures. The cells are plated onto Amp plates containing X-gal for blue-white selection and positive clones are screened and selected to prepare plasmid DNA. The clone is digested with appropriate matching restriction enzymes to release insert. For example, to facilitate cloning of the product, both 5′ and 3′ primers also introduced the restriction sites BglII and HindIII respectively to the final PCR product. A similar digestion also is used for subsequent PAV3 RHE gene expression plasmid. The PCR amplified product comprising of gene of interest is then cloned into the BglII and HindIII sites of the expression cassette within the PAV3 RHE plasmid. The recombinant PAV3 RHE plasmid and PAV3 LHE plasmid are then linearized using restriction enzyme which cut specifically within the plasmid backbone sequence but not within PAV3 genomic sequence or the inserted DNA. The digests are separated on a gel and purify cut insert and cut vector.

These inserts are used in subsequent ligation reactions. The DNA is transformed into E. coli TOP10 cells and colonies are picked and tested for the presence of the gene insert by PCR colony screens or preparation of DNA minipreps and restriction enzyme digests. The clone plasmid DNA is further sequenced to further verify sequence accuracy.

The linearized PAV3 LHE and PAV3 RHE plasmid DNA which both carry portions of the PAV3 viral genome were co-transfected into porcine cells (swine testis) cells. Both DNA fragments have an ˜1 kb region of homologous overlapping PAV3 sequence which directs homologous recombination to occur and reconstitute a competent full length recombinant PAV3 viral genome with the inserted DNA. Once PAV3 RHE plasmid constructs have been made and sequence verified, they are co-transfected into swine testis (ST) cells with PAV3 LHE plasmid using Lipofectamine 2000CD reagent (Invitrogen) to generate recombinant virus. Successive passage (e.g., success passage for 3-4 times) of transfected cells results in the enrichment of infective particles which appear as viral plaques. These represent recombinant PAV3 viruses expressing the gene of interest with a 5′ in frame signal sequence.

The virus stock is then propagated to prepare seed stocks of the recombinant virus. Further confirmation studies are performed to confirm that the viral genome is as expected by performing PCR screens and clone the gene expression cassette to sequence this region of the virus. Once this has been confirmed, the virus is expanded and once an appropriate titer is reached, the virus is used for further testing in animal trials.

### Example 2

In order to test the efficacy of the vaccines of the present invention, groups of piglets were given doses of the vaccine compositions described herein and the susceptibility of the pigs to a challenge with PRRS virus determined. In addition, the ability of the modified vaccine to induce neutralising antibody and to give protection when administered by the oral route will be tested.

45 pigs were randomly sorted into 3 equal size groups. The pigs were challenged with PRRSV (strain VR-2332 isolate BIAH-001 and were split into three treatment groups. Pigs in treatment group 1 (T1, mock vaccinated) were housed in a separate room to pigs in groups T2 (IM vaccinated) and T3 (oral/nasal vaccinated). T2 and T3 were housed in separate pens. All air in the rooms was HEPA filtered.

FIG. 2 shows the time line of the bleeding and testing of the pigs. All animals were observed daily for general health for the entire duration of the Study. The primary clinical parameters assessed were: depression; lethargy, respiratory rate, respiratory distress, moribund, death. On day 32 one of the pigs from group 1 (pig 983 unvaccinated) was observed as depressed and lethargic. Between days 33 and 40 the pig was repeatedly observed as lethargic and depressed. On day 40 it was observed as depressed, lethargic and moribund and was euthanized. No other pigs showed showed any clinical signs.

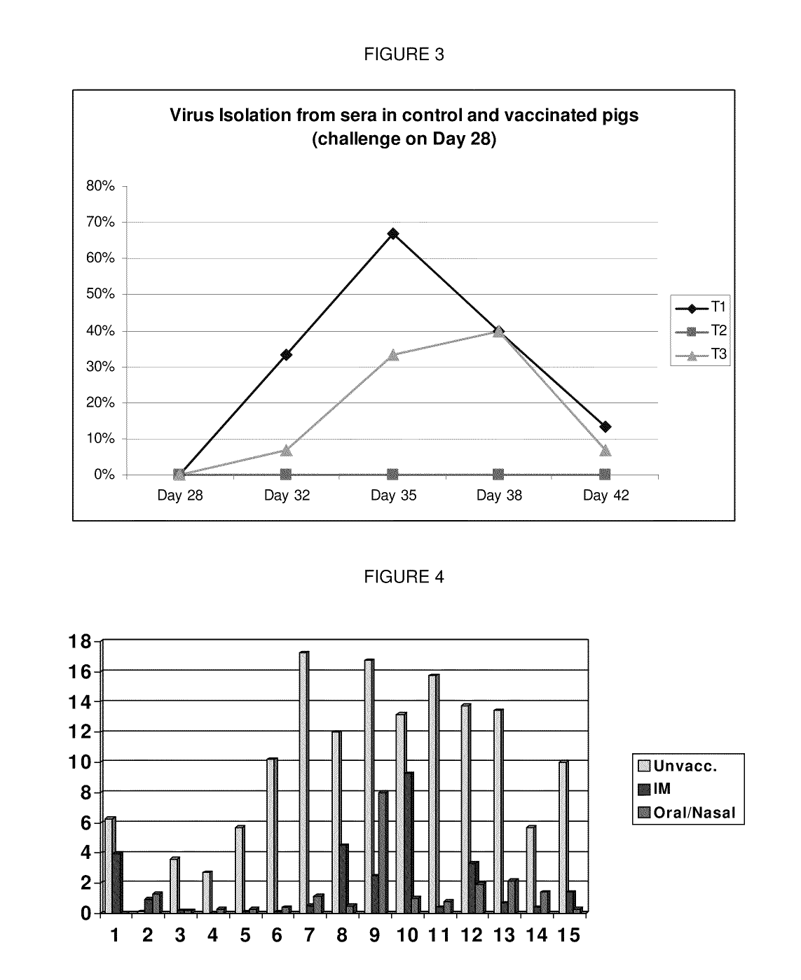

FIG. 3 shows virus isolation from the three test groups and clearly shows that oral/nasal administration of the vaccine was the most effective with IM administration also showing a significant reduction in percentage viremia as compared to the unvaccinated group T1.

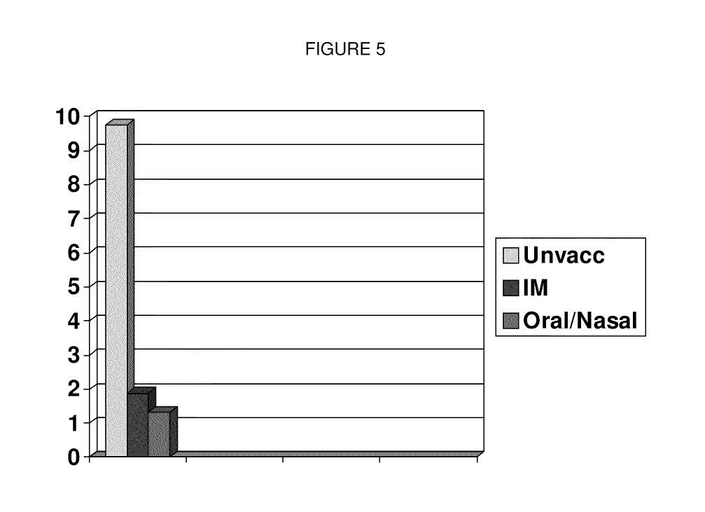

The individual pig lung scores (FIG. 4) and average lung scores (FIG. 5) are consistent with a demonstration that both IM administration and oral/nasal delivery of the vaccines of the invention produced a significant decrease in total lung pathology as compared to the unvaccinated group T1.

1. A replication competent porcine adenovirus type 3 virus (PADV3) comprising a heterologous nucleic acid that encodes a fusion of PRRS virus ORF6 and ORF5, inserted into a non-essential site of the PADV3 wherein said ORF5 is a modified ORF5 that contains a spacer sequence to separate the neutralizing and non-neutralizing epitopes encoded by ORF5 wherein the sequence of the nucleic acid encoding the ORF6ORF5m is the sequence of SEQ ID NO:1 or SEQ ID NO:2, wherein the spacer sequence encodes a Pan DR T-helper cell epitope (PADRES) as encoded by a sequence GCTAAATTTGTCGCAGCCTGGACTCTTAAGGCAGCGGCT (SEQ ID NO:22) or the sequence of GCTAAATTTGTCGCAGCCTGGACTCTTAAGGCAGCGGCT (SEQ ID NO:22) in SEQ ID NO:1 or SEQ ID NO:2 is replaced by any other nucleic acid sequence that encodes a peptide of between 10 to 15 amino acids in length.

2. The replication competent PADV3 of claim 1 wherein said non-essential site is selected from the group consisting the E3 region, ORF 1-2 and 4-7 of E4, and the region between map units 97-99.5 of the PADV3 genome.

3. The replication competent PADV3 of claim 2, wherein said non-essential site is the E3 region and said E3 region of said PADV3 is deleted and replaced with said nucleic acid that encodes the ORF6ORF5m.

4. The replication competent PADV3 of claim 2, wherein said non-essential site is the region between map units 97-99.5 of PADV3 genome and said nucleic acid that encodes the ORF6ORF5m is inserted into said region without deletion of the PADV3 map units 97-99.5.

5. The replication competent PADV3 of claim 2, wherein said non-essential site is the region between map units 97-99.5 of the PADV3 genome and said region between map units 97-99.5 of the PADV3 genome is deleted and replaced with said nucleic acid that encodes the ORF6ORF5m.

6. The replication competent PADV3 of claim 1 wherein said virus further comprises a nucleic acid encoding PRRS ORF7 inserted into either the E3 region or the region between map units 97-99.5 of the porcine adenovirus 3 vector.

7. The replication competent PADV3 of claim 1 further comprising a nucleic acid that encodes another antigen for eliciting an immune response in pigs.

8. The replication competent PADV3 of claim 1, wherein said nucleic acid sequence encodes a fusion protein having the sequence of SEQ ID NO:3 or SEQ ID NO:4.

9. The replication competent PADV3 of claim 6 wherein the PRRS ORF7 is encoded by a nucleic acid of SEQ ID NO:18.

10. The replication competent PADV3 of claim 9 wherein the ORF7 is encoded by SEQ ID NO:20.

11. A composition comprising a first replication competent PADV3 of claim 1, and a second recombinant expression vector that comprises an additional antigen for eliciting an immune response in pigs.

12. A vaccine for eliciting a protective response against PRRSV infection in pigs comprising a veterinarily acceptable vehicle or excipient and a replication competent PADV3 of claim 1, wherein said vaccine elicits neutralizing antibodies against PRRSV within two weeks of administration to a pig.

13. The vaccine of claim 12, further comprising one or more additional antigen for vaccination of pigs wherein said additional one or more antigen is provided as a protein component in the veterinarily acceptable vehicle or excipient of said vaccine.

14. A vaccine for the protection of pigs against diseases caused by PRRSV, said vaccine comprising a recombinant PADV3 virus vector comprising a heterologous nucleic acid that encodes a fusion of PRRS virus ORF6 and ORF5, inserted into a non-essential site of the PADV3 wherein said ORF5 is a modified ORF5 that contains a spacer sequence to separate the neutralizing and non-neutralizing epitopes encoded by ORF5 wherein the sequence of the nucleic acid encoding the ORF6ORF5m is the sequence of SEQ ID NO:1 or SEQ ID NO:2, wherein the spacer sequence encodes a Pan DR T-helper cell epitope (PADRES) as encoded by a sequence GCTAAATTTGTCGCAGCCTGGACTCTTAAGGCAGCGGCT (SEQ ID NO:22) or the sequence of GCTAAATTTGTCGCAGCCTGGACTCTTAAGGCAGCGGCT (SEQ ID NO:22) in SEQ ID NO:1 or SEQ ID NO:2 is replaced by any other nucleic acid sequence that encodes a peptide of between 10 to 15 amino acids in length.

15. The vaccine of claim 14, wherein said non-essential site is the E3 region and said E3 region of said PADV3 is deleted and replaced with said nucleic acid that encodes the ORF6ORF5m.

16. The vaccine of claim 14, wherein said non-essential site is the region between map units 97-99.5 of PADV3 genome and said nucleic acid that encodes the ORF6ORF5m is inserted into said region without deletion of the PADV3 map units 97-99.5.

17. The vaccine of claim 14, wherein said non-essential site is the region between map units 97-99.5 of the PADV3 genome and said region between map units 97-99.5 of the PADV3 genome is deleted and replaced with said nucleic acid that encodes the ORF6ORF5m.

18. The vaccine of claim 14, wherein said PADV3 further comprises a nucleic acid encoding PRRS ORF7 inserted into either the E3 region or the region between map units 97-99.5 of the porcine adenovirus 3 vector.

19. A vaccine for eliciting a protective response against PRRSV infection in pigs comprising a composition of claim 11.

20. The vaccine of claim 12 or claim 14 wherein said vaccine is formulated for aerosol administration.

21. The vaccine of claim 12 or claim 14 wherein said vaccine is formulated for oral, nasal, intramuscular, subcutaneous, or intradermal delivery.

22. A method of immunizing a pig against PRRSV comprising administering to said pig a vaccine of claim 12 or claim 14, wherein said immunization increases the presence of neutralizing antibodies against PRRSV in said pig within two weeks of the first administration of said vaccine to said pig.

23. An expression construct comprising a CMV promoter operatively linked to a nucleic acid that encodes an ORF6 fused to a modified ORF5 wherein the modified ORF5 has been modified to spatially separate the neutralizing and non-neutralizing epitopes, wherein said expression construct further comprises a nucleic acid that encodes PRRS ORF7 operatively linked to a major late promoter and said ORF5m encoding sequence and said ORF7 sequence comprise a polyA flanking sequence.

24. The expression construct of claim 23, wherein said ORF6 sequence has a nucleic acid sequence of SEQ ID NO:6 (lelystad), SEQ ID NO:9 (consensus) or SEQ ID NO:15 (asain).

25. The expression construct of claim 23, wherein said ORF5m sequence has a nucleic acid sequence of SEQ ID NO:14 (asian construct) or SEQ ID NO:11 (consensus).

26. The expression construct of claim 23, wherein said expression construct is a bicistronic construct in which a sequence of SEQ ID NO:17 encodes the ORF6OR5m fusion and a sequence of SEQ ID NO:20 encodes the ORF7.

27. A recombinant PADV3 that comprises an expression construct of claim 23.

28. A vaccine for eliciting a protective response against PRRSV infection in pigs comprising a recombinant PADV3 of claim 27.


SEQUENCE LISTING (23 sequences)

SEQ ID NO: 1 - DNA, 1173 bp, Artificial
  ORF6ORF5m encoding sequence from 49D4 including ORF5 from Asian strain
atggggtcgtccttagatgacttctgtcatgatagcacggctccagaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcacggtttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaagttaccaccatgttggggaaatgcttgaccgcgggctgttgctcgcaattgctttttttgtggtgtatcgtgccgttcttttctgctgtgcccgccagcgccaacgctaaatttgtcgcagccttgactcttaaggcagcggctagaaacaacagctcccatttacagctgatttacaacttgacgctatgtgagctgaatggcacagattggctagctggcaaatttgattgggcagtggagagttttgtcatctttcccgttttgactcacattgtctcctatggtgccctcactactagccatttccttgacacagccgctttagtcactgtgtctaccgccggttttcttcacgggcggtatgtcctaagcagcatctacgctgtctgtgccctggctgcgttgacttgcttcgtcattaggtttgcaaagaattgcatgtcctggcgctacgcgtgcaccagatacaccaactttcttctggacactaagggcagactctatcggtggcggtcgcctgtcatcatagagagaaggggcaaagttgaggtcgaaggtcatctgatcgacctcaaaagagttgtgcttgatggttccgtggcaacccctgtaaccagagtttcagcggaacaatggggtcgtccttag

SEQ ID NO: 2 - DNA, 1173 bp, Artificial
  ORF6ORF5m encoding sequence from Consensus Lelystad ORF6
atgggaagcctagacgatttttgcaacgatcctaccgccgcacaaaagctcgtgctagcctttagcatcacatacacacctataatgatatacgcccttaaggtgtcacgcggccgactcctggggctgttgcacatcctaatatttctgaactgttcctttacattcggatacatgacatatgtgcattttcaatccactaaccgtgtcgcacttaccctgggggctgttgtcgcccttctgtggggtgtttacagcttcacagagtcatggaagtttatcacttccagatgcagattgtgttgccttggccggcgatacattctggcccctgcccatcacgtagaaagtgctgcaggtctccattcaatcccagcgtctggtaaccgagcatacgctgtgagaaagcccggactaacatcagtgaacggcactctagtaccaggacttcggagcctcgtgctgggcggcaaacgagctgttaaacgaggagtggttaacctcgtcaagtatggccggggtaccaccatgagatgttctcacaaattggggcgtttcttgactccgcactcttgcttctggtggctttttttgctgtgtaccggcttgtcctggtcctttgccgatgctaaatttgtcgcagcctggactcttaaggcagcggctggcaacggcaacagctcgacataccaatacatatataacttgacgatatgcgagctgaatgggaccgactggttgtccagccattttggttgggcagtcgagacctttgtgctttacccggttgccactcatatcctctcactgggttttctcacaacaagccatttttttgacgcgctcggtctcggcgctgtatccactgcaggatttgttggcgggcggtacgtactctccagcgtctacggcgcttgtgctttcgcagcgttcgtatgttttgtcatccgtgctgctaaaaattgcatggcctgccgctatgcccgtacccggtttaccaacttcattgtggacgaccgggggagagttcatcgatggaagtctccaatagtggtagaaaaattgggcaaagccgaagtcgatggcaacctcgtcaccatcaaacatgtcgtcctcgaaggggttaaagctcaacccttgacgaggacttcggctgagcaatgggaggcctag

SEQ ID NO: 3 - protein, 390 aa, Artificial
  Amino acid sequence of ORF6ORF5m from Asian Strain
MGSSLDDFCHDSTAPEKVLLAFSITYTPVMIYALKVSRGRLLGLLHLLIFLNCAFTFGYMTFAHFQSTNKVALTMGAVVALLWGVYSAIETWKFITSRCRLCLLGRKYILAPAHHVESAARFHPIAANDNHAFVVRRPGSTTVNGTLVPGLKSLVLGGRKAVKQGVVNLVKYAKVTTMLGKCLTAGCCSQLLFLWCIVPFFSAVPASANAKFVAALTLKAAARNNSSHLQLIYNLTLCELNGTDWLAGKFDWAVESFVIFPVLTHIVSYGALTTSHFLDTAALVTVSTAGFLHGRYVLSSIYAVCALAALTCFVIRFAKNCMSWRYACTRYTNFLLDTKGRLYRWRSPVIIERRGKVEVEGHLIDLKRVVLDGSVATPVTRVSAEQWGRP

SEQ ID NO: 4 - protein, 390 aa, Artificial
  Amino acid sequence of ORF6ORF5m from Lelystad
MGSLDDFCNDPTAAQKLVLAFSITYTPIMIYALKVSRGRLLGLLHILIFLNCSFTFGYMTYVHFQSTNRVALTLGAVVALLWGVYSFTESWKFITSRCRLCCLGRRYILAPAHHVESAAGLHSIPASGNRAYAVRKPGLTSVNGTLVPGLRSLVLGGKRAVKRGVVNLVKYGRGTTMRCSHKLGRFLTPHSCFWWLFLLCTGLSWSFADAKFVAAWTLKAAAGNGNSSTYQYIYNLTICELNGTDWLSSHFGWAVETFVLYPVATHILSLGFLTTSHFFDALGLGAVSTAGFVGGRYVLSSVYGACAFAAFVCFVIRAAKNCMACRYARTRFTNFIVDDRGRVHRWKSPIVVEKLGKAEVDGNLVTIKHVVLEGVKAQPLTRTSAEQWEA

SEQ ID NO: 5 - DNA, 603 bp, Artificial
  ORF5 of Asian strain of PRRS Virus
atgttggggaaatgcttgaccgcgggctgttgctcgcaattgctttttttgtggtgtatcgtgccgttcttttctgctgtgcccgccagcgccaacagaaacaacagctcccatttacagctgatttacaacttgacgctatgtgagctgaatggcacagattggctagctggcaaatttgattgggcagtggagagttttgtcatctttcccgttttgactcacattgtctcctatggtgccctcactactagccatttccttgacacagccgctttagtcactgtgtctaccgccggttttcttcacgggcggtatgtcctaagcagcatctacgctgtctgtgccctggctgcgttgacttgcttcgttattaggtttgcaaagaattgcatgtcctggcgctacgcgtgcaccagatacaccaactttcttctggacactaagggcagactctatcggtggcggtcgcctgtcatcatagagaaaaggggcaaagttgaggtcgaaggtcatctgatcgacctcaaaagagttgtgcttgatggttccgtggcaacccctgtaaccagagtttcagcggaacaatggggtcgtccttag

SEQ ID NO: 6 - protein, 199 aa, Artificial
  Amino acid sequence of ORF5 of Asian strain of PRRS Virus
MLGKCLTAGCCSQLLFLWCIVPFFSAVPASANRNNSSHLQLIYNLTLCELNGTDWLAGKFDWAVESFVIFPVLTHIVSYGALTTSHFLDTAALVTVSTAGFLGRYVLSSIYAVCALAALTCFVIRFAKNCMSWRYACTRYTNFLLDTKGRLYRWRSPVIIEKRGKVEVEGHLIDLKRVVLDGSVATPVTRVSAEQWGRP

SEQ ID NO: 7 - DNA, 606 bp, Artificial
  ORF 5 of Lelystad strain
atgagatgttctcacaaattggggcgtttcttgactccgcactcttgcttctggtggctttttttgctgtgtaccggcttgtcctggtcctttgccgatggcaacggcgacagctcgacataccaatacatatataacttgacgatatgcgagctgaatgggaccgactggttgtccagccattttggttgggcagtcgagacctttgtgctttacccggttgccactcatatcctctcactgggttttctcacaacaagccatttttttgacgcgctcggtctcggcgctgtatccactgcaggatttgttggcgggcggtacgtactctgcagcgtctacggcgcttgtgctttcgcagcgttcgtatgttttgtcatccgtgctgctaaaaattgcatggcctaccgctatgcccgtacccggtttaccaacttcattgtggacgaccgggggagagttcatcgatggaagtctccaatagtggtagaaaaattgggcaaagccgaagtcgatggcaacctcgtcaccatcaaacatgtcgtcctcgaaggggttaaagctcaacccttgacgaggacttcggctgagcaatgggaggcctag

SEQ ID NO: 8 - DNA, 522 bp, Artificial
  ORF 6 Lelystad
atgggaggcctagacgatttttgcaacgatcctatcgccgcacaaaagctcgtgctagcctttagcatcacatacacacctataatgatatacgcccttaaggtgtcacgcggccgactcctggggctgttgcacatcctaatatttctgaactgttcctttacattcggatacatgacatatgtgcattttcaatccactaaccgtgtcgcacttaccctgggggctgttgtcgcccttctgtggggtgtttacagcttcacagagtcatggaagtttatcacttccagatgcagattgtgttgccttggccggcgatacattctggcccctgcccatcacgtagaaagtgctgcaggtctccattcaatctcagcgtctggtaaccgagcatacgctgtgagaaagcccggactaacatcagtgaacggcactctagtaccaggacttcggagcctcgtgctgggcggcaaacgagctgttaaacgaggagtggttaacctcgtcaagtatggccggtaa

SEQ ID NO: 9 - DNA, 528 bp, Artificial
  ORF6 Consensus and Linker added
atgggaagcctagacgatttttgcaacgatcctaccgccgcacaaaagctcgtgctagcctttagcatcacatacacacctataatgatatacgcccttaaggtgtcacgcggccgactcctggggctgttgcacatcctaatatttctgaactgttcctttacattcggatacatgacatatgtgcattttcaatccactaaccgtgtcgcacttaccctgggggctgttgtcgcccttctgtggggtgtttacagcttcacagagtcatggaagtttatcacttccagatgcagattgtgttgccttggccggcgatacattctggcccctgcccatcacgtagaaagtgctgcaggtctccattcaatcccagcgtctggtaaccgagcatacgctgtgagaaagcccggactaacatcagtgaacggcactctagtaccaggacttcggagcctcgtgctgggcggcaaacgagctgttaaacgaggagtggttaacctcgtcaagtatggccgggttaccacc

SEQ ID NO: 10 - protein, 176 aa, Artificial
  Amino acid Sequence of Consensus ORF6 with added linker
MGSLDDFCNDPTAAQKLVLAFSITYTPIMIYALKVSRGRLLGLLHILIFLNCSFTFGYMTYVHFQSTNRVALTLGAVVALLWGVYSFTESWKFITSRCRLCCLGRRYILAPAHHVESAAGLHSIPASGNRAYAVRKPGLTSVNGTLVPGLRSLVLGGKRAVKRGVVNLVKYGRGTT

SEQ ID NO: 11 - DNA, 645 bp, Artificial
  ORF5 Consensus and Modified
atgagatgttctcacaaattggggcgtttcttgactccgcactcttgcttctggtggctttttttgctgtgtaccggcttgtcctggtcctttgccgatgctaaatttgtcgcagcctggactcttaaggcagcggctggcaacggcaacagctcgacataccaatacatatataacttgacgatatgcgagctgaatgggaccgactggttgtccagccattttggttgggcagtcgagacctttgtgctttacccggttgccactcatatcctctcactgggttttctcacaacaagccatttttttgacgcgctcggtctcggcgctgtatccactgcaggatttgttggcgggcggtacgtactctccagcgtctacggcgcttgtgctttcgcagcgttcgtatgttttgtcatccgtgctgctaaaaattgcatggcctgccgctatgcccgtacccggtttaccaacttcattgtggacgaccgggggagagttcatcgatggaagtctccaatagtggtagaaaaattgggcaaagccgaagtcgatggcaacctcgtcaccatcaaacatgtcgtcctcgaaggggttaaagctcaacccttgacgaggacttcggctgagcaatgggaggcctag

SEQ ID NO: 12 - protein, 214 aa, Artificial
  Amino acid sequence of Consensus sequence for modified ORF5
MRCSHKLGRFLTPHSCFWWLFLLCTGLSWSFADAKFVAAWTLKAAAGNGNSSTYQYIYNLTICELNGTDWLSSHFGWAVETFVLYPVATHILSLGFLTTSHFFDALGLGAVSTAGFVGGRYVLSSVYGACAFAAFVCFVIRAAKNCMACRYARTRFTNFIVDDRGRVHRWKSPIVVEKLGKAEVDGNLVTIKHVVLEGVKAQPLTRTSAEQWEA

SEQ ID NO: 13 - DNA, 642 bp, Artificial
  Nucleic acid sequence of Asian Strain ORF5m
atgttggggaaatgcttgaccgcgggctgttgctcgcaattgctttttttgtggtgtatcgtgccgttcttttctgctgtgcccgccagcgccaacgctaaatttgtcgcagccttgactcttaaggcagcggctagaaacaacagctcccatttacagctgatttacaacttgacgctatgtgagctgaatggcacagattggctagctggcaaatttgattgggcagtggagagttttgtcatctttcccgttttgactcacattgtctcctatggtgccctcactactagccatttccttgacacagccgctttagtcactgtgtctaccgccggttttcttcacgggcggtatgtcctaagcagcatctacgctgtctgtgccctggctgcgttgacttgcttcgtcattaggtttgcaaagaattgcatgtcctggcgctacgcgtgcaccagatacaccaactttcttctggacactaagggcagactctatcggtggcggtcgcctgtcatcatagagagaaggggcaaagttgaggtcgaaggtcatctgatcgacctcaaaagagttgtgcttgatggttccgtggcaacccctgtaaccagagtttcagcggaacaatggggtcgtccttag

SEQ ID NO: 14 - protein, 213 aa, Artificial
  Amino acid sequence of Asian Strain ORF5m
MLGKCLTAGCCSQLLFLWCIVPFFSAVPASANAKFVAALTLKAAARNNSSHLQLIYNLTLCELNGTDWLAGKFDWAVESFVIFPVLTHIVSYGALTTSHFLDTAALVTVSTAGFLHGRYVLSSIYAVCALAALTCFVIRFAKNCMSWRYACTRYTNFLLDTKGRLYRWRSPVIIERRGKVEVEGHLIDLKRVVLDGSVATPVTRVSAEQWGRP

SEQ ID NO: 15 - DNA, 520 bp, Artificial
  Nucleic acid sequence of ORF6 in Asian construct
atggggtcgtccttagatgacttctgtcatgatagcacggctccagaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcacggtttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgcca

SEQ ID NO: 16 - protein, 173 aa, Artificial
  Amino acid sequence of ORF6 in Asian construct
MGSSLDDFCHDSTAPEKVLLAFSITYTPVMIYALKVSRGRLLGLLHLLIFLNCAFTFGYMTFAHFQSTNKVALTMGAVVALLWGVYSAIETWKFITSRCRLCLLGRKYILAPAHHVESAARFHPIAANDNHAFVVRRPGSTTVNGTLVPGLKSLVLGGRKAVKQGVVNLVKYA

SEQ ID NO: 17 - DNA, 2423 bp, Artificial
  CMVp-ORF6-5Mod-pA
accgacgcctacctgacgctgcagaactccagctcccttccccgcagcggcggcatcggcagcgagcaatttgtccgcgagttcgtgcccacggtctacatcaaccccttctccggaccgcccgggacctaccccgaccagttcatcgccaactacaacatcctaacggactctgtagcaggctatgactgacggtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcgatcgcccaccatggggtcgtccttagatgacttctgtcatgatagcacggctccagaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcacggtttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaagttaccaccatgttggggaaatgcttgaccgcgggctgttgctcgcaattgctttttttgtggtgtatcgtgccgttcttttctgctgtgcccgccagcgccaacgctaaatttgtcgcagccttgactcttaaggcagcggctagaaacaacagctcccatttacagctgatttacaacttgacgctatgtgagctgaatggcacagattggctagctggcaaatttgattgggcagtggagagttttgtcatctttcccgttttgactcacattgtctcctatggtgccctcactactagccatttccttgacacagccgctttagtcactgtgtctaccgccggttttcttcacgggcggtatgtcctaagcagcatctacgctgtctgtgccctggctgcgttgacttgcttcgtcattaggtttgcaaagaattgcatgtcctggcgctacgcgtgcaccagatacaccaactttcttctggacactaagggcagactctatcggtggcggtcgcctgtcatcatagagagaaggggcaaagttgaggtcgaaggtcatctgatcgacctcaaaagagttgtgcttgatggttccgtggcaacccctgtaaccagagtttcagcggaacaatggggtcgtccttagcccctgcaggtaagtcatgatatcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctaggcctgttgtacagatgaagtagctccccctctttcccattcccccatttttctctattcaataaagttgcttacctgagttcatccacactcgctctgccagtgcagtcagcagcccgat

SEQ ID NO: 18 - DNA, 372 bp, Artificial
  ORF7 Nucleic acid
atgccaaataacaacggcaggcagcagaagagaaagaagggggatggccagccagtcaatcagctgtgccagatgctgggtaagatcatcgctcagcaaaaccagtccagaggcaagggaccgggaaagaaaattaagaagaaaagcccggagaagccccattttcctctagcgactgaagatgatgtcagacatcactttacccctagtgagcggcaattgtgtctgtcgtcaatccagaccgcttttaatcaaggtgctgggacttgcaccctgtcagattcagggaggataagttacactgtggagtttagtttgcctacgcatcatactgtgcgcttaattcgcgtcacagcatcaccctcagcatga

SEQ ID NO: 19 - protein, 123 aa, Artificial
  ORF7 amino acid
MPNNNGRQQKRKKGDGQPVNQLCQMLGKIIAQQNQSRGKGPGKKIKKKSPEKPHFPLATEDDVRHHFTPSERQLCLSSIQTAFNQGAGTCTLSDSGRISYTVEFSLPTHHTVRLIRVTASPSA

SEQ ID NO: 20 - DNA, 1066 bp, Artificial
  MLP-ORF7-pA
gcggtcgttgggcactaatggcgaggcctgctcgagcgtgtggagacagaggtcctcgtcgtccgcgtccaggaagtggattggtcgccagtggtagtccacgtgaccggcttgcgggtcggggggtataaaaggcgcgggccggggtgcgtggccgtcagttgcttcgcaggcctcgtcaccggagtccgcgtctccggcgtctcgcgctgcggctgcatctgtggtcccggagtcttcaggtccttgttgaggaggtactcctgatcgctgtcccagtacttggcgtgtgggaagccgtcctgatcgcgtacctcctgctgttgcagcgcttcggcaaacacgcgcacctgctcttcggacccggcgaagcgttcgacgaaggcgtctagccagcaacagtcgcaagcctgcagatctggatccaccatgccaaataacaacggcaggcagcagaagagaaagaagggggatggccagccagtcaatcagctgtgccagatgctgggtaagatcatcgctcagcaaaaccagtccagaggcaagggaccgggaaagaaaattaagaagaaaagcccggagaagccccattttcctctagcgactgaagatgatgtcagacatcactttacccctagtgagcggcaattgtgtctgtcgtcaatccagaccgcttttaatcaaggtgctgggacttgcaccctgtcagattcagggaggataagttacactgtggagtttagtttgcctacgcatcatactgtgcgcttaattcgcgtcacagcatcaccctcagcatgaagcttggatccgttgttgtatacttgtttattgcagcttataatggttacaaataaagcaatagcatcacaaatttcacaaataaagcatttttttcactgcattctagttgtggtttgtccaaactcatcaatgtatcttatcatgtctgagctcgaattgggaattcccgccagccgggctccgccccgcgacctgcggactttgaccccgcccctcggactttgaccgttcccacgccacgttattttcccacgcgacgtca

SEQ ID NO: 21 - DNA, 606 bp, Artificial
  Consensus ORF 5 nucleic acid sequence without PADRE sequence
atgagatgttctcacaaattggggcgtttcttgactccgcactcttgcttctggtggctttttttgctgtgtaccggcttgtcctggtcctttgccgatgctaacggcaacagctcgacataccaatacatatataacttgacgatatgcgagctgaatgggaccgactggttgtccagccattttggttgggcagtcgagacctttgtgctttacccggttgccactcatatcctctcactgggttttctcacaacaagccatttttttgacgcgctcggtctcggcgctgtatccactgcaggatttgttggcgggcggtacgtactctccagcgtctacggcgcttgtgctttcgcagcgttcgtatgttttgtcatccgtgctgctaaaaattgcatggcctgccgctatgcccgtacccggtttaccaacttcattgtggacgaccgggggagagttcatcgatggaagtctccaatagtggtagaaaaattgggcaaagccgaagtcgatggcaacctcgtcaccatcaaacatgtcgtcctcgaaggggttaaagctcaacccttgacgaggacttcggctgagcaatgggaggcctag

SEQ ID NO: 22 - DNA, 39 bp, Artificial
  PADRE Sequence
gctaaatttgtcgcagcctggactcttaaggcagcggct

SEQ ID NO: 23 - protein, 13 aa, Artificial
  Amino Acid PADRE sequence
AKFVAALTLKAAA